(19) United States
(12) Patent Application Publication
(10) Pub. No.: US 2023/0348951 A1
(43) Pub. Date: Nov. 2, 2023

(54) DIAGNOSING AND TREATING CHRONIC KIDNEY DISEASE IN A FELINE

(71) Applicant: Société des Produits Nestlé S.A., Vevey (CH)

(72) Inventor: Qinghong Li, Chesterfield, MO (US)

(21) Appl. No.: 18/194,406

(22) Filed: Mar. 31, 2023

Related U.S. Application Data
(60) Provisional application No. 63/335,973, filed on Apr. 28, 2022.

(51) Int. Cl. C12Q 1/10; C12Q 1/14

(52) U.S. Cl. CPC C12Q 1/10; C12Q 1/14; G01N 2800/347; G01N 2800/56

(57) ABSTRACT

The present invention relates to methods for diagnosing chronic kidney disease in a feline, including early-stage chronic kidney disease, by using microbiome including specific genera and species. In one embodiment, the method can comprise measuring an absolute abundance of fecal bacteria including Faecalibacterium, Turicibacter, Streptococcus, Bifidobacterium, Bacteroides, E. coli, and C. hiranonis; calculating a dysbiosis index based on the fecal bacteria; and determining that the feline has chronic kidney disease if the dysbiosis index is greater than or equal to 0.5.

### CROSS REFERENCE TO RELATED APPLICATION

This application claims the benefit of and priority to U.S. Provisional Application Ser. No. 63/335,973 filed Apr. 28, 2022, the disclosure of which is incorporated in its entirety herein by this reference.

### BACKGROUND

The kidneys have five primary functions. They filter waste products from the body (e.g., urea and creatinine), regulate electrolytes (e.g., potassium, calcium, phosphorus, and sodium), produce erythropoietin (which stimulates bone marrow to produce red blood cells), produce rennin (which controls blood pressure), and produce and concentrate urine.

Chronic kidney disease (“CKD”) is a progressive kidney disease which has four phases: loss of renal reserve, renal insufficiency, azotemia, and uremia. The kidneys have a large built-in reserve as only approximately 30% of kidney capacity is needed for normal kidney function. Kidney capacity diminishes with time for a variety of reasons, for example, advanced age and diseases and medications that damage the kidney(s). Renal insufficiency is characterized by decrease in renal function and is generally observed when about 70% of kidney function has been lost (i.e., when only about 30% of kidney capacity is available). Clinical signs are typically not obvious during the stages of loss of renal reserve and renal insufficiency, thereby making it difficult to detect CKD. Stage 1 CKD is nonazotemic and generally cannot be diagnosed as there are no overt clinical symptoms. Stage 2 CKD is mildly azotemic with clinical signs absent or mild. Stage 3 is moderately azotemic with clinical signs present. Stage 4 is severely azotemic with clinical signs present.

CKD is a terminal disease and is one of the leading causes of death in felines. Thus, there is a need for compositions and methods for diagnosing and preventing CKD in felines, especially diagnosing early-stage CKD. There is also a need for compositions and methods for treating CKD which provide partial or complete relief.

### SUMMARY

The present disclosure relates generally to diagnosing chronic kidney disease (CKD) in a feline, and in one aspect, early-stage CKD. In one embodiment, a method of diagnosing CKD in a feline can comprise measuring an absolute abundance of fecal bacteria including Faecalibacterium, Turicibacter, Streptococcus, Bifidobacterium, Bacteroides, E. coli, and C. hiranonis, calculating a dysbiosis index based on the fecal bacteria, and determining that the feline has CKD if the dysbiosis index is greater than or equal to 0.5.

In another embodiment, a method of diagnosing early-stage CKD in a feline can comprise measuring an absolute abundance of a biomarker selected from the group consisting of: Lactobacillus animalis, Subdoligranulum variabile, Catenibacterium mitsuokai, Collinsella aerofaciens, Ruminococcus obeum, Eubacterium biforme, Lactobacillus reuteri, Bifidobacterium pseudocatenulatum, Coprococcus comes, Megasphaera elsdenii, Lachnospiraceae bacterium_1_1_57FAA, Faecalibacterium prausnitzii, Eubacterium hallii, Ruminococcaceae bacterium_D16, Dorea longicatena, Clostridium hiranonis, Acidaminococcus fermentans, Saccharomyces cerevisiae, Streptococcus parauberis, Acidaminococcus intestini, Helicobacter canis, Bacteroides coprocola, and combinations thereof, and determining that the feline has early-stage CKD if the absolute abundance of the biomarker is within the ranges described in Table 5.

In still another embodiment, a method of diagnosing early-stage CKD in a feline can comprise measuring an absolute abundance of bacteria in a genus, wherein the genus is selected from the group consisting of Catenibacterium, Lactobacillus, Coprococcus, Megasphaera, Helicobacter, Eubacterium, Faecalibacterium, Acidaminococcus, Bifidobacterium, Subdoligranulum, Allobaculum, Escherichia, Enterococcus, and combinations thereof, and determining that the feline has early-stage CKD if the absolute abundance of the biomarker is within the ranges described in Table 3.

Additionally, a method of enabling treatment or slowing progression of CKD in a feline can comprise diagnosing CKD in the feline as disclosed herein and recommending a composition for the feline, wherein the composition treats or slows the progression of CKD in the feline.

Further, a method of enabling treatment or slowing progression of early-stage CKD in a feline can comprise diagnosing early-stage CKD in the feline as disclosed herein and recommending a composition for the feline, wherein the composition treats or slows the progression of early-stage CKD in the feline.

Additional features and advantages are described herein and will be apparent from the following Detailed Description

### DETAILED DESCRIPTION

### Definitions

As used in this disclosure and the appended claims, the singular forms “a,” “an” and “the” include plural referents unless the context clearly dictates otherwise. Thus, for example, reference to “a fecal bacteria or bacterium” or “the fecal bacteria or bacterium” includes two or more such bacteria or bacterium. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative, and are not exclusive or comprehensive.

As used herein, “about” is understood to refer to numbers in a range of numerals, for example the range of −10% to +10% of the referenced number, within −5% to +5% of the referenced number, or in one aspect, within −1% to +1% of the referenced number, and in a specific aspect, within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein, “between” is inclusive of the endpoints. For example, a dysbiosis index between 0.5 and 1.2 includes where the dysbiosis is 0.5 or 1.2.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed. When reference is made to the pH, values correspond to pH measured at 25° C. with standard equipment. An “amount” can be the total amount of the referenced component per serving of the composition or per distinct unit of the composition and/or can be the weight percentage of the referenced component by dry weight. Moreover, an “amount” includes zero; for example, the recitation of an amount of a compound does not necessarily mean that the compound is present, unless followed by a range that excludes zero.

As used herein, “absolute abundance” refers to the amount of each microorganism calculated by logarithm to the base 10 of the bacterial DNA abundance, which was measured by the quantitative PCR (qPCR) assay.

As used herein, “chronic kidney disease” or “CKD” refers to the persistent loss of kidney function over time in felines.

As used herein, “early-stage chronic kidney disease” refers to stage 1 or 2 chronic kidney disease (CKD) or CKD1/2 using the International Renal Interest Society (IRIS) guidelines (http://www.iris-kidney.com/guidelines/).

As used herein, “late-stage chronic kidney disease” refers to stage 3 or 4 chronic kidney disease (CKD) or CKD3/4 using the International Renal Interest Society (IRIS) guidelines (http://www.iris-kidney.com/guidelines/).

As used herein “dysbiosis index” or “DI” is calculated using the qPCR cycle threshold (Ct) values obtained for each bacterial group. To overcome variability between samples, the Ct values of each individual bacterial group were normalized by dividing them by the Ct values for total bacteria. The DI was established by the nearest centroid classifier algorithm, DI is defined as the difference between (Euclidean distance between the test sample and the healthy class centroid) and the (Euclidean distance between the test and the diseased class centroid). DI is calculated mathematically as follows: the DI of a text sample z is defined as:

DI(z;μ_{C}_{_{D}},μ_{C}_{_{H}})=∥z−μ_{C}_{_{H}}∥₂−∥z−μ_{C}_{_{D}}∥₂

Where μ_{CD} and μ_{C}_{_{H}} stand for the centroid of the diseased and healthy samples in the training set, respectively. The method for calculating feline DI is also described in “Dysbiosis index to evaluate the fecal microbiota in healthy cats and cats with chronic enteropathies” by Sung et al, Journal of Feline Medicine and Surgery, March 2022. doi:10.1177/1098612X221077876.

The methods disclosed herein may lack any step that is not specifically disclosed herein. Thus, a disclosure of an embodiment using the term “comprising” includes a disclosure of embodiments “consisting essentially of” and “consisting of” the steps identified. Any embodiment disclosed herein can be combined with any other embodiment disclosed herein unless explicitly and directly stated otherwise.

Unless defined otherwise, all technical and scientific terms and any acronyms used herein have the same meanings as commonly understood by one of ordinary skill in the art in the field of the invention. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

### EMBODIMENTS

The present inventor has discovered that chronic kidney disease (CKD) can be diagnosed based on specific microbiome including a dysbiosis index as well as specific genera and species. Further, the inventor has discovered that the present microbiome can be used to diagnose early-stage CKD in a feline. Such methods allow for an inexpensive and efficient diagnosis for conditions that can be difficult to diagnose as well as costly.

In one embodiment, a method of diagnosing CKD in a feline can comprise measuring an absolute abundance of fecal bacteria including Faecalibacterium, Turicibacter, Streptococcus, Bifidobacterium, Bacteroides, E. coli, and C. hiranonis, calculating a dysbiosis index based on the fecal bacteria, and determining that the feline has CKD if the dysbiosis index is greater than or equal to 0.5. Additionally, the method can also include measuring other bacteria. In one aspect, the fecal bacteria can include Blautia. In another aspect, the fecal bacteria can include Fusobacterium.

Generally, the feline can be diagnosed with CKD if the dysbiosis index is greater than or equal to 0.5. However, in one aspect, the determining step can also include determining that the feline has early-stage CKD when the dysbiosis index is between 0.5 and 1.2. Additionally, in another aspect, the present methods can further determine that the feline has late-stage CKD when the dysbiosis index is greater than 1.2.

In another embodiment, a method of diagnosing early-stage CKD in a feline can comprise measuring an absolute abundance of a biomarker selected from the group consisting of: Lactobacillus animalis, Subdoligranulum variabile, Catenibacterium mitsuokai, Collinsella aerofaciens, Ruminococcus obeum, Eubacterium biforme, Lactobacillus reuteri, Bifidobacterium pseudocatenulatum, Coprococcus comes, Megasphaera elsdenii, Lachnospiraceae bacterium_1_1_57FAA, Faecalibacterium prausnitzii, Eubacterium hallii, Ruminococcaceae bacterium_D16, Dorea longicatena, Clostridium hiranonis, Acidaminococcus fermentans, Saccharomyces cerevisiae, Streptococcus parauberis, Acidaminococcus intestini, Helicobacter canis, Bacteroides coprocola, and combinations thereof, and determining that the feline has early-stage CKD if the absolute abundance of the biomarker is within the ranges described in Table 5.

Generally, the feline can be diagnosed with early-stage CKD using various biomarkers, i.e., bacteria, discussed herein having specific absolute abundances. However, in one aspect, the diagnosis can be based at least two biomarkers. In another aspect, the diagnosis can be based on at least three biomarkers. In still another aspect, the diagnosis can be based on at least four biomarkers. In yet other aspects, the diagnosis can be based on five biomarkers, six biomarkers, seven biomarkers, eight biomarkers, nine biomarkers, ten biomarkers, fifteen biomarkers, or more.

In still another embodiment, a method of diagnosing early-stage CKD in a feline can comprise measuring an absolute abundance of bacteria in a genus, wherein the genus is selected from the group consisting of Catenibacterium, Lactobacillus, Coprococcus, Megasphaera, Helicobacter, Eubacterium, Faecalibacterium, Acidaminococcus, Bifidobacterium, Subdoligranulum, Allobaculum, Escherichia, Enterococcus, and combinations thereof, and determining that the feline has early-stage CKD if the absolute abundance of the biomarker is within the ranges described in Table 3.

Generally, the feline can be diagnosed with early-stage CKD using bacteria in various genera discussed herein having specific absolute abundances. However, in one aspect, the diagnosis can be based at least two genera. In another aspect, the diagnosis can be based on at least three genera. In still another aspect, the diagnosis can be based on at least four genera. In another aspect, the diagnosis can be based on at least five genera. In yet other aspects, the diagnosis can be based on six genera, seven genera, eight genera, nine genera, ten genera, or more.

Additionally, a method of enabling treatment or slowing progression of CKD in a feline can comprise diagnosing CKD in the feline as disclosed herein and recommending a composition for the feline, wherein the composition treats or slows the progression of CKD in the feline.

Further, a method of enabling treatment or slowing progression of early-stage CKD in a feline can comprise diagnosing early-stage CKD in the feline as disclosed herein and recommending a composition for the feline, wherein the composition treats or slows the progression of early-stage CKD in the feline.

Compositions useful for treating CKD including early-stage CKD include pet foods, supplements, and the like. Such compositions can include medium chain triglycerides, omega-3 fatty acids, Vitamin E, Vitamin C, B vitamins (including thiamin, riboflavin, pantothenic acid, niacin, pyridoxine, folic acid, biotin, and cobalamin), L-arginine, or sulfur-containing amino acids such as taurine. The composition can be a pet food, such as a wet pet food, a semi-moist pet food, or a dry pet food, e.g., kibble.

Generally, the medium chain triglycerides can be about 0.5 wt % to about 60 wt % of the composition. In one aspect, the medium chain triglycerides can be from about 1 wt % to about 20 wt % of the composition. In other aspects, the medium chain triglycerides can be from about 1 wt % to about 15 wt %, from about 1 wt % to about 10 wt %, or from about 2 wt % to about 10 wt % of the composition. The medium chain triglycerides may be prepared by any known process, such as direct esterification, rearrangement, fractionation and/or transesterification. For example, the medium chain triglycerides may be prepared from a source of vegetable oil, such as coconut oil, through a rearrangement process. The chain length and distribution thereof may vary depending on the source oil. For example, MCTs containing 1-10% C6, 30-60% C8, 30-60% C10 and 1-10% C12 can be derived from palm oil and/or coconut oil; in some embodiments, at least a portion of the MCTs are provided by coconut oil, but in other embodiments the composition does not contain coconut oil. MCTs containing at least about 95% C8 can be made by semi-synthetic esterification of octanoic acid to glycerin; in some embodiments thereof, the remainder of the fatty acids are C6 and C10. Mixtures comprising MCTs with about 50% total C8 and/or about 50% total C10 are also useful herein.

Non-limiting examples of suitable omega-3 fatty acids include eicosapentaenoic acid (EPA), docosahexaenoic acid (DHA), alpha-linolenic acid (ALA) and mixtures thereof. In one embodiment, the omega-3 fatty acids can range from about 0.2 wt % to about 3 wt % of the composition. In some embodiments, the omega-3 fatty acids are at least about 0.2 wt %, at least about 1.0 wt %, or at least about 2.0 wt %.

Non-limiting examples of suitable sulfur-containing amino acids include methionine, cysteine, homocysteine, taurine and mixtures thereof.

In an embodiment, the composition further comprises (i) carnitine, (ii) lysine and methionine, (iii) an antioxidant such as glutathione, or (iv) mixtures thereof. The composition can be high in protein, for example at least about 20 wt %, at least about 25 wt %, or even at least about 30 wt % of the composition. Additionally, the composition can have balanced amounts of magnesium, sodium and potassium; for example, the ratio of potassium to sodium can be about 5:1 to about 1:1, in one aspect, about 5:1 to about 2:1, with the magnesium in an amount of about 0.08 wt % to about 0.25 wt %, and in one aspect, from about 0.10 wt % to about 0.15 wt %. At least a portion of the magnesium, sodium and potassium can be provided as isolated compounds (e.g., salts). Alternatively or additionally, at least a portion of the magnesium, sodium and potassium can be provided by one or more foodstuffs. For example, magnesium can be provided by wheat bran, whole grains, leafy green vegetables, meat, beans and bananas; and potassium and sodium can be provided by meats, fish, whole grains, yogurt, bananas, sweet potatoes, squash, beans and tomatoes.

In an embodiment, the pet food composition provides complete nutrition as defined by the Association of American Feed Control Officials (AAFCO) and which depends on the type of animal for which the composition is intended (e.g., a cat). In another embodiment, the composition can be a supplement. Such a supplement can be added to a food composition or be administered in conjunction with a food composition, or administered separately.

The pet food composition can comprise meat, such as emulsified meat. Examples of suitable meat include poultry, beef, pork, lamb and fish, especially those types of meats suitable for pets. The meat can include any additional parts of an animal including offal. Some or all of the meat can be provided as one or more meat meals, namely meat that has been dried and ground to form substantially uniform-sized particles and as defined by AAFCO. Additionally or alternatively, vegetable protein can be used, such as pea protein, corn protein (e.g., ground corn or corn gluten), wheat protein (e.g., ground wheat or wheat gluten), soy protein (e.g., soybean meal, soy concentrate, or soy isolate), rice protein (e.g., ground rice or rice gluten) and the like.

The pet food compositions disclosed herein can comprise one or more of a vegetable oil, a flavorant, a colorant or water. Non-limiting examples of suitable vegetable oils include soybean oil, corn oil, cottonseed oil, sunflower oil, canola oil, peanut oil, safflower oil and the like. In some embodiments, the lipids in the composition can consist of the MCTs and one or more of any vegetable oil, any fish oil, the lipid from any meat, and any omega-3 fatty acids.

Non-limiting examples of suitable flavorants include yeast, tallow, rendered animal meals (e.g., poultry, beef, lamb, pork), flavor extracts or blends (e.g., grilled beef), animal digests, and the like. Non-limiting examples of suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The pet food compositions disclosed herein can optionally include additional ingredients, such as starches, humectants, oral care ingredients, preservatives, amino acids, fibers, prebiotics, sugars, animal oils, aromas, other oils additionally or alternatively to vegetable oil, salts, vitamins, minerals, probiotic microorganisms, bioactive molecules or combinations thereof.

Non-limiting examples of suitable starches include a grain such as corn, rice, wheat, barley, oats, potatoes, peas, beans, cassava, and the like, and mixtures of these grains, and can be included at least partially in any flour. Non-limiting examples of suitable humectants include salt, sugars, propylene glycol and polyhydric glycols such as glycerin and sorbitol, and the like. Non-limiting examples of suitable oral care ingredients include alfalfa nutrient concentrate containing chlorophyll, sodium bicarbonate, phosphates (e.g., tricalcium phosphate, acid pyrophosphates, tetrasodium pyrophosphate, metaphosphates, and orthophosphates), peppermint, cloves, parsley, ginger and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

Further, the composition, in whole or in part, can have a specific protein to phosphorous ratio between about 5:1 to about 15:1. The ratio can provide a palatability enhancing effect for renal diets and can be present in the basal or in a coating applied to the basal (e.g., a kibble). Other compositions can include protein, fat, fiber, carbohydrate, and optionally, a functional ingredient that reduces renal damage or enhances kidney function including those that modulate or decrease levels of circulating catabolites, modulate or decrease levels of phosphorous, decrease blood urea nitrogen levels (BUN) or BUN/creatine ratio levels. Functional ingredients include, but are not limited, to a conjugated linoleic acid. Generally, with respect to the pet food product, the functional ingredient acts to mitigate adverse effects of high protein diets. A natural or a synthetic functional ingredient is contemplated. Synthetic and semi-synthetic (i.e., isomerization of vegetable oil using, for example, rumen bacterium Butyrivibrio fibrisolvens) preparations of conjugated linoleic acid have been described and are considered suitable for the present invention (see, for example, U.S. Pat. Nos. 6,410,761; 6,380,409; and 5,554,646, each of which is herein incorporated by reference in their entirety). In such embodiments that the functional ingredient is a conjugated linoleic acid, the amount may be calculated as part of either the functional ingredient content, or of the fat content.

### EXAMPLES

The following non-limiting examples are illustrative of embodiments of the present disclosure.

### Example 1—CKD Study of Felines

Twenty-eight cats with no renal disease (healthy control), twenty-one cats with the IRIS stage 1 or 2 chronic kidney disease (CKD), and nine cats with stage 3 or 4 CKD were enrolled in the study. Cats were diagnosed and staged using the IRIS guidelines (http://www.iris-kidney.com/guidelines/).

Dysbiosis Index (DI)

The abundances of 10 bacterial groups, including total bacteria, Faecalibacterium, Turicibacter, Escherichia coli, Streptococcus, Blautia, Fusobacterium, Clostridium hiranonis, Bifidobacterium, and Bacteroides were evaluated on the fecal DNA samples from the three groups of cats with or without CKD. The qPCR primer sets, protocol and the method for dysbiosis index (DI) were described previously in Sung et al. Journal of Feline Medicine and Surgery, March 2022. A DI<0.5 indicates normobiosis, whereas a DI>=0.5 indicates dysbiosis.

Metagenomics Sequencing

The Illumina DNA library prep kit uses a bead-based transposome complex to tagment genomic DNA, which is a process that fragments DNA and then tags the DNA with adapter sequences in one step. After it is saturated with input DNA, the bead-based transposome complex fragments a set number of DNA molecules. This fragmentation provides flexibility to use a wide DNA input range to generate normalized libraries of consistent fragment size. Following tagmentation, a limited-cycle PCR adds Illumina DNA Prep-specific index adapter sequences to the ends of a DNA fragment. A subsequent Sample Purification Beads (SPB) cleanup step then purifies libraries for use on an Illumina sequencer. The double-stranded DNA library is denatured before hybridization of the biotin probe oligonucleotide pool. A total of 100 ng of DNA for each sample was used, following the user guide without any modifications: Illumina DNA Prep Reference Guide (Illumina DNA Prep Reference Guide (1000000025416)). The quality control and the quantification for the pooling was based on Qubit Hs dsDNA Kit quantification and quality check for few samples on the TapeStation with HSD5000 kit.

Two PE-150 sequencing runs were performed on NextSeq2000 with P3-300 cycles chemistry and loaded at 750 pM for the 1^{st} run and 650 pM for the second run and with 2% Phix. The sequencing runs were demultiplexed with DRAGEN on the NextSeq2000.

Bioinformatics and Statistical Analyses

The sequencing data from the two runs were combined and paired-end sequences were merged using the software PEAR (https://cme.hits.org/exelixis/web/software/pear/)_Paired-End read merger. Low quality sequences were trimmed using the software Trimmomatic (http://www.usadellab.org/cms/?page=trimmomatic), a flexible read trimming tool for Illumina sequencing data. The host genomic DNA sequences were separated and removed from the microbial genomic DNA sequences using the software KneadData (https://huttenhower.sph.harvard.edu/kneaddata/). Microbial profiling was performed using MetaPhlAn2 (https://huttenhower.sph.harvard.edu/metaphlan2/#:˜:text=MetaPhlAn%202.0, from %20metagenomic%20shotgun%20sequencing%20data.), Metagenomic Phylogenetic Analysis. The microbial compositional data were compared among the three groups of cats, non-CKD, CKD1/2, and CKD3/4, using the non-parametric Kruskal-wallis test, and the mean for each group was calculated.

Results

Table 1 provides total bacteria and bacterial genera/species with differential abundances among groups non-CKD, CKD stage1 or 2, and CKD stage 3 or 4, with a dysbiosis index for each category.

TABLE 1

| | Mean | Mean | Mean |
|---|---|---|---|
| | Non-CKD | CKD1/2 | CKD3/4 |
| Dysbiosis Index | 0.0444 | 0.9478 | 1.3883 |
| Total bacteria | 11.2027 | 11.0473 | 10.7939 |
| Faecalibacterium | 7.3714 | 6.8993 | 6.3835 |
| Turicibacter | 5.508 | 5.141 | 5.3491 |
| Streptococcus | 5.0834 | 4.9685 | 4.767 |
| E. coli | 4.7516 | 5.6724 | 6.2466 |
| Blautia | 10.3313 | 9.8959 | 9.8107 |
| Fusobacterium | 7.6022 | 7.7167 | 8.1719 |
| Clostridium hiranonis | 6.0066 | 4.9575 | 4.3646 |
| Bifidobacterium | 5.9813 | 5.5526 | 4.9606 |
| Bacteroides | 6.103 | 6.2331 | 6.0439 |

Table 2 provides bacterial genus with differential abundances among non-CKD, CKD stage1 or 2, and CKD stage 3 or 4 groups.

TABLE 2

| | | | |
|---|---|---|---|
| | Mean | Mean | Mean |
| Genus | Non-CKD | CKD1/2 | CKD3/4 |

Decreases in abundances

| | | | |
|---|---|---|---|
| Catenibacterium | 2.892 | 0.855 | 0.265 |
| Lactobacillus | 19.296 | 5.522 | 2.551 |
| Coprococcus | 0.151 | 0.024 | 0.01 |
| Megasphaera | 2.169 | 0.893 | 0.565 |
| Helicobacter | 0.023 | 0.001 | 0.256 |
| Eubacterium | 0.378 | 0.053 | 0.004 |
| Faecalibacterium | 0.069 | 0.016 | 0.012 |
| Acidaminococcus | 0.118 | 0.053 | 0.013 |
| Bifidobacterium | 8.656 | 10.338 | 2.485 |
| Subdoligranulum | 2.769 | 1.658 | 1.159 |

Increases in abundances

| | | | |
|---|---|---|---|
| Allobaculum | 0.022 | 0.501 | 4.162 |
| Escherichia | 1.99 | 4.879 | 8.909 |
| Enterococcus | 1.506 | 3.333 | 7.79 |

Table 3 provides diagnostic ranges for differentiating normal, early-stage CKD, and late-stage CKD disease states of cats based on the genera of Table 2.

TABLE 3

| | | | |
|---|---|---|---|
| | Mean Non-CKD | Mean | Mean CKD3/4 |
| | # is greater than | CKD1/2 | # is less than |
| Genus | (>) | # is between | (<) |

Decreases in abundances

| | | | |
|---|---|---|---|
| Catenibacterium | 1.874 | 1.874-0.56 | 0.56 |
| Lactobacillus | 12.409 | 12.409-4.037 | 4.037 |
| Coprococcus | 0.088 | 0.088-0.017 | 0.017 |
| Megasphaera | 1.531 | 1.531-0.729 | 0.729 |
| Helicobacter | 0.012 | 0.012-0.129 | 0.129 |
| Eubacterium | 0.216 | 0.216-0.029 | 0.029 |
| Faecalibacterium | 0.043 | 0.043-0.014 | 0.014 |
| Acidaminococcus | 0.086 | 0.086-0.033 | 0.033 |
| Bifidobacterium | 9.497 | 9.497-6.412 | 6.412 |
| Subdoligranulum | 2.214 | 2.214-1.409 | 1.409 |

Increases in abundances

| | Mean Non-CKD | Mean | Mean CKD3/4 |
|---|---|---|---|
| | # is less than | CKD1/2 | # is greater than |
| Genus | (<) | # is between | (>) |
| Allobaculum | 0.262 | 0.262-2.332 | 2.332 |
| Escherichia | 3.435 | 3.435-6.894 | 6.894 |
| Enterococcus | 2.42 | 2.42-5.562 | 5.562 |

Table 4 provides bacterial genus species with differential abundances among non-CKD, CKD stage1 or 2, and CKD stage 3 or 4 groups.

TABLE 4

| | | | | |
|---|---|---|---|---|
| | | Mean | Mean | Mean |
| Genus | Species | Non-CKD | CKD1/2 | CKD3/4 |

Decreases in abundances

| | | | | |
|---|---|---|---|---|
| Lactobacillus | animalis | 14.793 | 0.023 | 0.542 |
| Subdoligranulum | variabile | 0.104 | 0.022 | 0.005 |
| Catenibacterium | mitsuokai | 2.892 | 0.855 | 0.265 |
| Collinsella | aerofaciens | 6.782 | 7.626 | 1.025 |
| Ruminococcus | obeum | 0.018 | 0.005 | 0.002 |
| Eubacterium | biforme | 7.468 | 3.3 | 2.224 |
| Lactobacillus | reuteri | 2.413 | 0.365 | 0.013 |
| Bifidobacterium | pseudocatenulatum | 1.69 | 0.375 | 0 |
| Coprococcus | comes | 0.151 | 0.024 | 0.01 |
| Megasphaera | elsdenii | 0.176 | 0.069 | 0.058 |
| Lachnospiraceae | bacterium_ | 1.019 | 0.425 | 1.382 |
| | 1_1_57FAA | | | |
| Faecalibacterium | prausnitzii | 0.069 | 0.016 | 0.012 |
| Eubacterium | hallii | 0.378 | 0.053 | 0.004 |
| Ruminococcaceae | bacterium_D16 | 0.545 | 0.107 | 0.296 |
| Dorea | longicatena | 0.58 | 0.039 | 0 |
| Clostridium | hiranonis | 7.335 | 5.899 | 3.09 |
| Acidaminococcus | fermentans | 0.06 | 0.009 | 0.001 |
| Saccharomyces | cerevisiae | 0.013 | 0.001 | 0 |
| Streptococcus | parauberis | 0.031 | 0.002 | 0.005 |
| Acidaminococcus | intestini | 0.058 | 0.043 | 0.012 |
| Helicobacter | canis | 0.013 | 0.001 | 0.254 |
| Bacteroides | coprocola | 0.267 | 0.03 | 0.509 |

Increases in abundances

| | | | | |
|---|---|---|---|---|
| Lachnospiraceae | bacterium_ | 0.003 | 0.039 | 0.268 |
| | 2_1_46FAA | | | |
| Enterococcus | avium | 0 | 0 | 0.2 |
| Ruminococcus | gnavus | 0.87 | 6.391 | 11.774 |
| Eubacterium | dolichum | 0.052 | 0.045 | 1.381 |
| Allobaculum | stercoricanis | 0.022 | 0.501 | 4.162 |

Table 5 provides diagnostic ranges for differentiating normal, early-stage CKD, and late-stage CKD disease states of cats based on the genus species of Table 4.

TABLE 5

| | | | | |
|---|---|---|---|---|
| | | Mean Non- | Mean | Mean |
| | | CKD | CKD1/2 | CKD3/4 |
| | | # is greater | # is | # is less |
| Genus | Species | than (>) | between | than (<) |

Decreases in abundances

| | | | | |
|---|---|---|---|---|
| Lactobacillus | animalis | 7.408 | 7.408- | 0.283 |
| | | | 0.283 | |
| Subdoligranulum | variabile | 0.063 | 0.063- | 0.014 |
| | | | 0.014 | |
| Catenibacterium | mitsuokai | 1.874 | 1.874- | 0.56 |
| | | | 0.56 | |
| Collinsella | aerofaciens | 7.204 | 7.204- | 4.326 |
| | | | 4.326 | |
| Ruminococcus | obeum | 0.012 | 0.012- | 0.004 |
| | | | 0.004 | |
| Eubacterium | biforme | 5.384 | 5.384- | 2.762 |
| | | | 2.762 | |
| Lactobacillus | reuteri | 1.389 | 1.389- | 0.189 |
| | | | 0.189 | |
| Bifidobacterium | pseudocatenulatum | 1.033 | 1.033- | 0.188 |
| | | | 0.188 | |
| Coprococcus | comes | 0.088 | 0.088- | 0.017 |
| | | | 0.017 | |
| Megasphaera | elsdenii | 0.123 | 0.123- | 0.064 |
| | | | 0.064 | |
| Lachnospiraceae | bacterium_ | 0.722 | 0.722- | 0.904 |
| | 1_1_57FAA | | 0.904 | |
| Faecalibacterium | prausnitzii | 0.043 | 0.043- | 0.014 |
| | | | 0.014 | |
| Eubacterium | hallii | 0.216 | 0.216- | 0.029 |
| | | | 0.029 | |
| Ruminococcaceae | bacterium_D16 | 0.326 | 0.326- | 0.202 |
| | | | 0.202 | |
| Dorea | longicatena | 0.31 | 0.31- | 0.02 |
| | | | 0.02 | |
| Clostridium | hiranonis | 6.617 | 6.617- | 4.495 |
| | | | 4.495 | |
| Acidaminococcus | fermentans | 0.035 | 0.035- | 0.005 |
| | | | 0.005 | |
| Saccharomyces | cerevisiae | 0.007 | 0.007- | 0.001 |
| | | | 0.001 | |
| Streptococcus | parauberis | 0.017 | 0.017- | 0.004 |
| | | | 0.004 | |
| Acidaminococcus | intestini | 0.051 | 0.051- | 0.028 |
| | | | 0.028 | |
| Helicobacter | canis | 0.007 | 0.007- | 0.128 |
| | | | 0.128 | |
| Bacteroides | coprocola | 0.149 | 0.149- | 0.27 |
| | | | 0.27 | |

Increases in abundances

| | | Mean Non- | Mean | Mean |
|---|---|---|---|---|
| | | CKD | CKD1/2 | CKD3/4 |
| | | # is less | # is | # is greater |
| Genus | Species | than (<) | between | than (>) |
| Lachnospiraceae | bacterium_ | 0.021 | 0.021- | 0.154 |
| | 2_1_46FAA | | 0.154 | |
| Enterococcus | avium | 0 | 0-0.1 | 0.1 |
| Ruminococcus | gnavus | 3.631 | 3.631- | 9.083 |
| | | | 9.083 | |
| Eubacterium | dolichum | 0.049 | 0.049- | 0.713 |
| | | | 0.713 | |
| Allobaculum | stercoricanis | 0.262 | 0.262- | 2.332 |
| | | | 2.332 | |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of diagnosing chronic kidney disease in a feline, comprising:
measuring an absolute abundance of fecal bacteria including Faecalibacterium, Turicibacter, Streptococcus, Bifidobacterium, Bacteroides, E. coli, and C. hiranonis;
calculating a dysbiosis index based on the fecal bacteria; and
determining that the feline has chronic kidney disease if the dysbiosis index is greater than or equal to 0.5.

2. The method of claim 1, wherein the fecal bacteria include Blautia.

3. The method of claim 1, wherein the fecal bacteria include Fusobacterium.

4. The method of claim 1, further determining that the feline has early-stage chronic kidney disease when the dysbiosis index is between 0.5 and 1.2.

5. The method of claim 1, further determining that the feline has late-stage chronic kidney disease when the dysbiosis index is greater than 1.2.

6. A method of diagnosing early-stage chronic kidney disease in a feline, comprising:
measuring an absolute abundance of a biomarker selected from the group consisting of: Lactobacillus animalis, Subdoligranulum variabile, Catenibacterium mitsuokai, Collinsella aerofaciens, Ruminococcus obeum, Eubacterium biforme, Lactobacillus reuteri, Bifidobacterium pseudocatenulatum, Coprococcus comes, Megasphaera elsdenii, Lachnospiraceae bacterium_1_1_57FAA, Faecalibacterium prausnitzii, Eubacterium hallii, Ruminococcaceae bacterium_D16, Dorea longicatena, Clostridium hiranonis, Acidaminococcus fermentans, Saccharomyces cerevisiae, Streptococcus parauberis, Acidaminococcus intestini, Helicobacter canis, Bacteroides coprocola, and combinations thereof; and
determining that the feline has early-stage chronic kidney disease if the absolute abundance of the biomarker is within the following ranges:

| | | |
|---|---|---|
| Lactobacillus | animalis | 7.408-0.283 |
| Subdoligranulum | variabile | 0.063-0.014 |
| Catenibacterium | mitsuokai | 1.874-0.56 |
| Collinsella | aerofaciens | 7.204-4.326 |
| Ruminococcus | obeum | 0.012-0.004 |
| Eubacterium | biforme | 5.384-2.762 |
| Lactobacillus | reuteri | 1.389-0.189 |
| Bifidobacterium | pseudocatenulatum | 1.033-0.188 |
| Coprococcus | comes | 0.088-0.017 |
| Megasphaera | elsdenii | 0.123-0.064 |
| Lachnospiraceae | bacterium_1_1_57FAA | 0.722-0.904 |
| Faecalibacterium | prausnitzii | 0.043-0.014 |
| Eubacterium | hallii | 0.216-0.029 |
| Ruminococcaceae | bacterium_D16 | 0.326-0.202 |
| Dorea | longicatena | 0.31-0.02 |
| Clostridium | hiranonis | 6.617-4.495 |
| Acidaminococcus | fermentans | 0.035-0.005 |
| Saccharomyces | cerevisiae | 0.007-0.001 |
| Streptococcus | parauberis | 0.017-0.004 |
| Acidaminococcus | intestini | 0.051-0.028 |
| Helicobacter | canis | 0.007-0.128 |
| Bacteroides | coprocola | 0.149-0.27 |
| Lachnospiraceae | bacterium_2_1_46FAA | 0.021-0.154 |
| Enterococcus | avium | 0-0.1 |
| Ruminococcus | gnavus | 3.631-9.083 |
| Eubacterium | dolichum | 0.049-0.713 |
| Allobaculum | stercoricanis | 0.262-2.332 |

7. The method of claim 6, wherein the determining is based on at least two biomarkers.

8. The method of claim 6, wherein the determining is based on at least three biomarkers.

9. The method of claim 6, wherein the determining is based on at least four biomarkers.

10. A method of diagnosing early-stage chronic kidney disease in a feline, comprising:
measuring an absolute abundance of bacteria in a genus, wherein the genus is selected from the group consisting of Catenibacterium, Lactobacillus, Coprococcus, Megasphaera, Helicobacter, Eubacterium, Faecalibacterium, Acidaminococcus, Bifidobacterium, Subdoligranulum, Allobaculum, Escherichia, Enterococcus, and combinations thereof; and
determining that the feline has early-stage chronic kidney disease if the absolute abundance of the biomarker is within the following ranges:

| | |
|---|---|
| Catenibacterium | 1.874-0.56 |
| Lactobacillus | 12.409-4.037 |
| Coprococcus | 0.088-0.017 |
| Megasphaera | 1.531-0.729 |
| Helicobacter | 0.012-0.129 |
| Eubacterium | 0.216-0.029 |
| Faecalibacterium | 0.043-0.014 |
| Acidaminococcus | 0.086-0.033 |
| Bifidobacterium | 9.497-6.412 |
| Subdoligranulum | 2.214-1.409 |
| Allobaculum | 0.262-2.332 |
| Escherichia | 3.435-6.894 |
| Enterococcus | 2.42-5.562 |

11. The method of claim 10, wherein the determining is based on at least two genera.

12. The method of claim 10, wherein the determining is based on at least three genera.

13. The method of claim 10, wherein the determining is based on at least four genera.

14. A method of enabling treatment or slowing progression of chronic kidney disease in a feline, comprising diagnosing chronic kidney disease (CKD) in the feline according to claim 1 and recommending a composition for the feline, wherein the composition treats or slows the progression of CKD in the feline.

15. A method of enabling treatment or slowing progression of early-stage chronic kidney disease in a feline, comprising diagnosing early-stage chronic kidney disease (CKD) in the feline according to claim 6 or claim 10 and recommending a composition for the feline, wherein the composition treats or slows the progression of early-stage CKD in the feline.
